(19) United States
(12) Patent Application Publication
(10) Pub. No.: US 2016/0264928 A1
(43) Pub. Date: Sep. 15, 2016

(54) System, Method, and Apparatus for Sterilization with Additional Nutrients for Microbiological Inoculation

(71) Applicant: MAGNEGAS CORPORATION, Tarpon Springs, FL (US)

(72) Inventor: Ermanno Santilli, Clearwater, FL (US)

(73) Assignee: MAGNEGAS CORPORATION, Tarpon Springs, FL (US)

(21) Appl. No.: 15/066,795

(22) Filed: Mar. 10, 2016

Related U.S. Application Data
(60) Provisional application No. 62/131,465, filed on Mar. 11, 2015.

(51) Int. Cl. C12N 1/20; A61L 2/14

(52) U.S. Cl. CPC C12N 1/20; A61L 2/14

(57) ABSTRACT

A method of processing excrement of mammals, fish, birds, reptiles, insects, and/or humans includes sterilizing the excrement to create a sterile fluid, adding one or more nutrients and adding microorganisms such as genus bacillus to the sterile fluid creating a processed fluid. The good microorganisms multiply and divide in the processed fluid, thereby reducing the growth of harmful or unwanted microorganisms in the processed fluid. The processed fluid is then, for example, released into waterways or used to water/fertilized crops.

### CROSS-REFERENCE TO RELATED APPLICATION

This application claims the benefit of U.S. provisional application No. 62/131,465 filed on Mar. 11, 2015, the disclosure of which is incorporated by reference.

### FIELD

This invention relates to the field of LIQUID processing and more particularly to a system for sterilizing a substrate then adding nutrients for the purpose of adding a beneficial biological agent.

### BACKGROUND

Management of biological excrement (e.g. manure, sewerage, etc.) is often difficult due to biological organisms residing in the excrement such as e-Coli, Coliform, etc. Several methods of processing biological excrement have been performed with various levels of success.

It should be noted that not all biological organisms are bad and that some are deemed good microorganisms that are beneficial to soil, plants, and livestock, etc.

One way to process biological excrement is to pasteurize the excrement. For example, the biological excrement is heated to a temperature that will kill the unwanted biological organisms. This is typically a temperature of between 60 C and 80 C, a temperature at which many organisms are killed. Unfortunately, at these temperatures, many beneficial organisms are eliminated while many detrimental organisms are not eliminated. Once the temperature recedes, the detrimental organisms again multiply.

Another way to process biological excrement is to sterilize the excrement by chemicals. After the excrement is sterilized by chemicals, the resulting material is unsuitable for land application due to the residual amounts of the chemical sterilizers.

In many situations, the neutralized material lacks sufficient nutrients, or at least some needed components, preventing growth and multiplication of beneficial biological cells.

What is needed is a system that will properly manage biological A liquid substrate, eliminating bad microorganisms and adding additional nutritional components to the neutralized the substrate, rendering the substrate ready for inoculation with good microorganisms.

### SUMMARY

In one embodiment, a system is disclosed including a sterilization device that kills most biological components of a substrate (e.g. biological excrement), after which, any nutritional components that are needed to grow good biology are added before or in conjunction with the addition of a good biology. The good biology has beneficial organisms that multiply within the sterilized and fertilized substrate, thereby preventing or reducing growth of detrimental organisms within the sterilized substrate.

In one embodiment, a method of processing excrement is disclosed including sterilizing the excrement to create a sterile fluid then adding nutrients and adding microorganisms to the sterile fluid creating a processed fluid. The microorganisms multiply and divide in the processed fluid, thereby reducing the growth of bad microorganisms in the processed fluid.

In another embodiment, an apparatus for processing excrement is disclosed including a pressure vessel containing in its interior excrement and at least one set of electrodes. An electric arc is formed between the electrodes. The excrement is exposed to a plasma of the electric arc thereby producing a sterilized fluid. The pressure vessel is fluidly interfaced to a fluid processing tank, such that after producing the sterilized fluid, the sterilized fluid flows into fluid processing tank. A first canister that has there within one or more nutrients is fluidly interfaced to the fluid processing tank for adding the one or more nutrients to the sterile fluid in the fluid processing tank by control of a first valve. A second canister having there within microorganisms is fluidly interfaced to the fluid processing tank for adding the microorganisms to the sterile fluid in the fluid processing tank by control of a second valve. The microorganisms consume the one or more nutrients and multiply and divide in the sterile fluid, thereby reducing the growth of bad microorganisms in the sterile fluid.

In another embodiment, a system for processing excrement is disclosed including a pressure vessel containing in its interior excrement (e.g., sewerage, animal waste) and at least one set of electrodes. An electric arc is formed between the electrodes and the excrement is exposed to a plasma of the electric arc thereby producing a sterilized fluid. There is, optionally, a system for collecting the gas and optionally a system for replenishing the excrement as the excrement is converted into the sterile fluid, both of which are interfaced to the pressure vessel. The sterilized fluid is deposited into a fluid processing tank and one or more nutrients are added to the sterile fluid in the fluid processing tank. Microorganisms are then added to the sterile fluid in the fluid processing tank so that the microorganisms consume the nutrients and multiply and divide in the sterile fluid, thereby reducing the growth of bad microorganisms in the sterile fluid.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a schematic view of an exemplary system for the processing of excrement.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

Dealing with biological excrement is a challenge, due to the fast growth of harmful, bad microorganisms within the nutrient-rich biological excrement. The following description describes a system and method for processing excrement of mammals, birds, reptiles, and/or humans. Although processing of excrement from any animal, bird, insect, reptile, etc., is anticipated, the described system and processes are anticipated for any liquid substrate such as standing water, river water, pond water, liquids produced during mining operations, etc. In addition, it is fully anticipated that the substrate will include dissolved or co-mixed solids as well, as would river water and sewerage, etc.

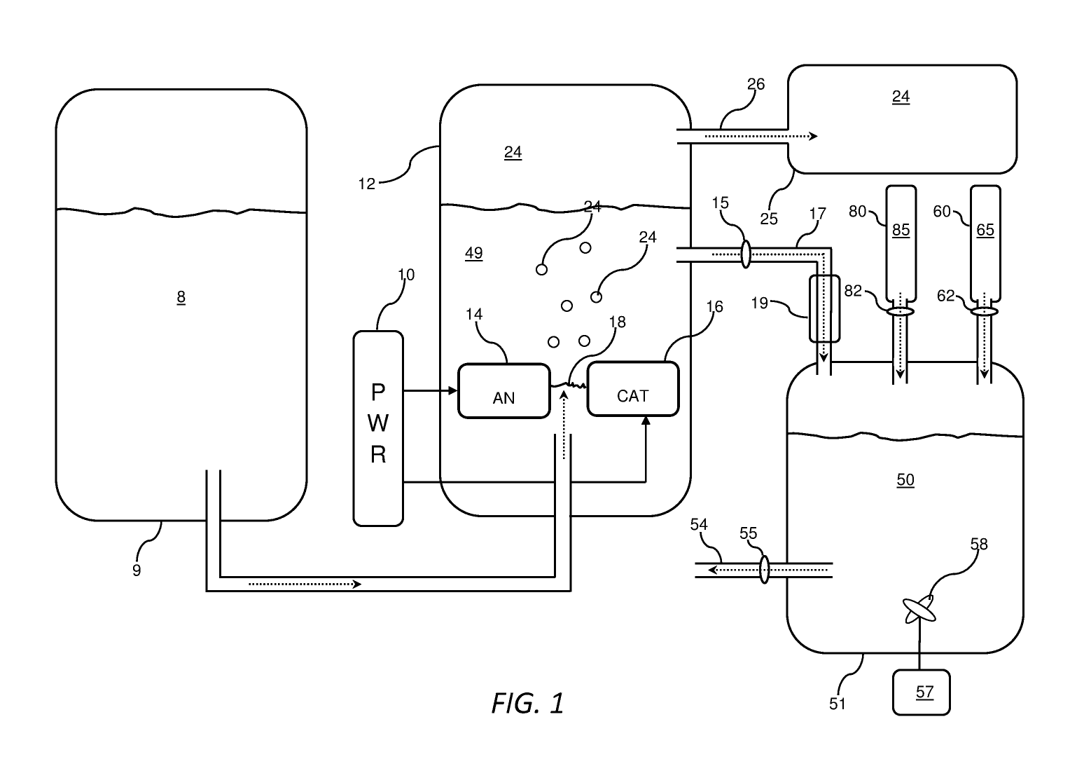

Referring to FIG. 1, an exemplary system for the processing excrement is shown. This is but an example of one system for the processing of excrement, as other such systems are also anticipated. As shown, a liquid is flown through a reactor as one way to sterilize a liquid. This is discussed in, for example, U.S. Pat. No. 7,780,924 issued Aug. 24, 2010, U.S. Pat. No. 6,183,604 issued Feb. 6, 2001, U.S. Pat. No. 6,540,966 issued Apr. 1, 2003, U.S. Pat. No. 6,972,118 issued dec. 6, 2005, U.S. Pat. No. 6,673,322 issued Jan. 6, 2004, U.S. Pat. No. 6,663,752 issued Dec. 16, 2003, U.S. Pat. No. 6,926,872 issued Aug. 9, 2005, and U.S. Pat. No. 8,236,150 issued Aug. 7, 2012, all of which are incorporated by reference. In this exemplary system, the sterilization of a liquid (e.g., excrement, manure, sewerage) is performed within the plasma 18 of a submerged electric arc, though other methods of sterilization are equally anticipated and included here within.

In this example, the excrement 8 is collected and stored in a tank 9. At some time, for example, when a certain amount of excrement 8 has been collected in the tank 9, the excrement (e.g., excrement from mammals, birds, fish, reptiles, humans, insects) is pumped into a reactor 12 and force to flow into a plasma 18, for example, a plasma 18 created by an arc struck between two electrodes 14/16. In some embodiments, the now sterile fluid 49 is circulated within the reactor 12 and back through the plasma 18 to further assure proper sterilization. The arc is powered by a source of electric power 10.

As a bi-product of the excrement 8 flowing through the plasma 18, a gas 24 is released, percolating to the top of the sterile fluid 49 and extracted through plumbing 26 into a gas holding tank 25. This gas burns at a very high temperature and is useful in welding and cutting of metals.

The now sterile fluid 49 has none or very small amounts of micro-organisms remaining, and in some reactors 12, the sterile fluid 49 is totally void of micro-organisms. Once the fluid 49 is finished in the sterilization process, the fluid 49 is sent into a fluid processing tank 51 by way of a valve 15, using a pump (not shown) or gravity to flow the fluid 49 from the reactor 12 into the fluid processing tank 51. In some embodiments, a chiller 19 cools the liquid so as not to destroy any biological material that is added in the fluid processing tank 51.

If the sterile fluid 49 was left alone, both good and bad microbes would begin to split and multiply. Without any external influence, these microbes multiply within the sterile fluid 49, but because of the rate of multiplication, the good microbes will not be as effective in overtaking the harmful microbes, and, therefore, the fluid is not sterile for long after cooling. The sterile fluid 49 after it is moved into the fluid processing tank 51 is referred to as processed fluid 50, as it will include both good microbes and dangerous microbes.

In some embodiments, the processed fluid 50 is void of ample nutrients for the growth of beneficial biology (cells); while in other embodiments the processed fluid 50 has certain nutrients but lacks other nutrients needed for the growth of beneficial biology (cells). In some embodiments, the processed fluid 50 is cooled to a suitable temperature for multiplication of microbes, by convection, conduction, or a chilling system.

Either before the optional cooling or after the optional cooling, one or more nutrients 85 or supplemental nutrients 85 are added to the processed fluid 50 in the processing tank 51 from one or more nutrient containers 80, through any known valve/injector system 82. Note that any mechanism for inserting/introducing the nutrient(s) 85 into the processing tank 51 is anticipated, including pouring the nutrients 85 into the fluid processing tank 51. In some embodiments, the liquid 50, now containing the added nutrients 85 is stirred or agitated, for example by a stirring blade 58 driven by a motor 57.

Normally, soil contains the micro biology named genus bacillus, which is known as a good microorganism, in that, it multiplies rapidly and attacks/kills bad microorganisms such as e-Coli and Coliform. As described above, leaving the processed fluid 50 alone will result in the bad microorganisms taking hold and multiplying in the processed fluid 50. Some genus bacillus will also take hold and multiply and attempt to kill the bad microorganisms, but the process is too slow and, due to the long period of time and extreme activity within this nutrient rich, processed fluid 50. There is too much opportunity for the bad microorganisms to multiply and mutation of either microorganism into even more dangerous microorganisms is possible.

To prevent the bad microorganisms from taking hold and multiplying in the processed fluid 50, a supply of good microorganisms 65 (e.g. including genus bacillus) stored in a container 60 is injected into the processed fluid 50 stored within the fluid processing tank 51 through, for example, a pump/valve/injector 62, possibly including a one-way valve to prevent the sterile fluid from contaminating the good microorganisms 65 stored in the container 60. Note that any mechanism for inserting/introducing the good microorganisms 65 into the processing tank 51 is anticipated, including pouring the good microorganisms 65 into the fluid processing tank 51. In some embodiments, the liquid 50, now containing the added nutrients 85 and the good microorganisms 65 is stirred or agitated, for example by the stirring blade 58 driven by the motor 57.

Sufficient good microorganisms 65 are added to the processed fluid 50 such that, the good microorganisms split/multiply at a fast enough rate as to overpower any bad microorganisms that may be introduced into the processed fluid 50, thereby the good microorganisms 65 will overpower any bad microorganisms that find their way into the processed fluid 50, which is now not sterile being that it contains the good microorganisms, but is safe to transform and use, because there are no bad microorganisms present.

A correct balance of nutrient(s) 85 is added to the processed fluid 50 to encourage growth of the good microorganisms 65. For example, if the sterile fluid 49 lacks a needed amount of oxygen, nitrogen, iron, phosphorus, etc., an amount of such is added to the processed fluid 50 to provide a suitable environment for proper growth and reproduction of the good microorganisms.

Once the good microorganisms 65 stabilize and maintain control of the processed fluid 50, some or all of the processed fluid 50 is removed from the tank 51 through plumbing 54, controlled by a pump/valve 55. The processed fluid 50 with beneficial microorganisms 65 is now used in many different applications such as crop watering/fertilizing or it is sent into the ground water or surface waterways.

The above process includes receiving an amount of excrement from mammals, birds, fish, reptiles, humans, insects, etc., and sterilizing the excrement, killing most or all microbes within the excrement, creating a sterile fluid 49. This sterile liquid contains some nutrients, but lacks at least one other nutrient (e.g., mineral, chemical, gas) that is needed for proper and rapid growth of good microbes 65. Therefore, one or more nutrients 85 are added to the sterile liquid 49, now a processed fluid 50 and, either before, during, or after the addition of the one or more nutrients 85, good microbes 85 are added to the processed fluid 50, such that, with the help of the one or more nutrients 85, the good microbes 85 find a suitable environment for growth and multiplication. If the sterilizing adds heat to the processed fluid 50, in some embodiments, the processed fluid 50 is cooled at any step of the above sequence, as needed for proper growth and multiplication of the good microbes 65.

Although sterilization of the excrement 8 is disclosed as passing the excrement through a plasma 18 created by an electric arc between two electrodes 14/18, it is fully anticipated that sterilization is performed in any known way, using any form of plasma or any alternate method of sterilization, as long as the sterilizing agent does not remain within the sterile fluid 49. For example, if sterilization is performed by adding certain chemicals, those chemicals remain within the sterile fluid 49 and will kill and/or prevent the good microorganisms from splitting/multiplying. If such chemicals are introduced, the sterile fluid 49 must be aerated to allow those chemicals to evaporate, but during the aeration process, bad microorganisms will again be introduced and contaminate what was once a sterile fluid 49 before the good microorganisms can be added. Although any form of sterilization is anticipated, sterilization by exposure to an electric arc, sterilization by exposure to a plasma, sterilization by heating, sterilization by pressurization, sterilization using ultraviolet light, sterilization by using infrared light, sterilization by microwave, sterilization by exposure to a magnetic field, sterilization by exposure to radiation, and sterilization by exposure to laser light, of any combination of forms of sterilization is anticipated.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A method of processing excrement comprising:
sterilizing excrement, creating a sterile fluid;
after sterilizing, adding one or more nutrients to the sterile fluid and adding good microorganisms to the sterile fluid creating a processed fluid such that the good microorganisms multiply and divide in the processed fluid, thereby reducing the growth of bad microorganisms in the processed fluid.

2. The method of claim 1, wherein the good microorganisms comprise genus bacillus.

3. The method of claim 1, wherein the excrement is excrement from a mammal.

4. The method of claim 3, wherein the mammal is a pig.

5. The method of claim 3, wherein the mammal is a human.

6. The method of claim 1, wherein the excrement is excrement from a fish.

7. The method of claim 1, wherein the excrement is excrement from a reptile.

8. The method of claim 1, wherein the excrement is excrement from a bird.

9. The method of claim 1, wherein the step of sterilizing allows for growth of the good microbiology by neutralizing existing or competitive bad micro biology.

10. The method of claim 1, wherein the step of sterilization includes eliminating bad microbiology prior to the step of adding good microbiology, thereby reducing possible mutation of the good microbiology.

11. The method of claim 1, wherein the step of sterilizing includes sterilizing with any process selected from the group consisting of heating, pressurizing, exposing to electric arcs, exposing to ultraviolet, exposing to infrared, through which bad microbiology is eliminated while generally retaining nutrients in the excrement.

12. The method of claim 1, wherein the step of sterilizing prior to adding good microorganisms reduces a cost of the step of adding good microorganisms.

13. The method of claim 1, further comprising a step of cooling prior to adding good microorganisms.

14. The method of claim 1, further comprising a step of mixing the sterile fluid for distributing the one or more nutrients in the sterile fluid.

15. A system for processing excrement comprising:
a pressure vessel containing in its interior an excrement and at least one set of electrodes;
an electric arc formed between the electrodes;
means for exposing the excrement to a plasma of the electric arc thereby producing a sterilized fluid and converting at least some of the excrement into a gas;
means for collecting the gas;
means for replenishing the excrement interfaced to the pressure vessel;
means for capturing the sterilized fluid into a fluid processing tank;
means for adding one or more nutrients to the sterile fluid in the fluid processing tank; and
means for adding microorganisms to the sterile fluid in the fluid processing tank;
whereas the microorganisms multiply and divide in the sterile fluid, thereby reducing the growth of bad microorganisms in the sterile fluid.

16. The system of claim 15, further comprising a chiller interfaced between the pressure vessel and the fluid processing tank for cooling the sterile fluid before the sterile fluid enters the fluid processing tank.

17. The method of claim 15, wherein the microorganisms comprise genus bacillus.

18. An apparatus for processing excrement comprising:
a pressure vessel containing in its interior an excrement and at least one set of electrodes;
an electric arc formed between the electrodes;
means for exposing the excrement to a plasma of the electric arc thereby producing a sterilized fluid;
an output of the pressure vessel fluidly interfaced to a fluid processing tank, such that after producing the sterilized fluid, the sterilized fluid flows into fluid processing tank;
a first canister having there within one or more nutrients fluidly interfaced to the fluid processing tank for adding the one or more nutrients to the sterile fluid in the fluid processing tank by control of a first valve; and
a second canister having there within microorganisms, the second canister fluidly interfaced to the fluid processing tank for adding the microorganisms to the sterile fluid in the fluid processing tank by control of a second valve;
whereas the microorganisms multiply and divide in the sterile fluid, thereby reducing the growth of bad microorganisms in the sterile fluid.

19. The apparatus of claim 18, further comprising a chiller interfaced between the pressure vessel and the fluid processing tank for cooling the sterile fluid before the sterile fluid enters the fluid processing tank.

20. The apparatus of claim 18, wherein the microorganisms comprise genus bacillus.
